(19) United States
(12) Patent Application Publication
(10) Pub. No.: US 2025/0090354 A1
(43) Pub. Date: Mar. 20, 2025

(54) METHOD FOR PRODUCING LIVING BODY INDWELLING OBJECT

(71) Applicant: TERUMO KABUSHIKI KAISHA, Tokyo (JP)

(72) Inventors: Shuhei MATSUSHITA, Hadano (JP); Yuhei MORI, Yamato (JP); Yuuji MOTOSE, Hiratsuka (JP); Tsutomu SUGIKI, Hadano (JP)

(73) Assignee: TERUMO KABUSHIKI KAISHA, Tokyo (JP)

(21) Appl. No.: 18/800,470

(22) Filed: Aug. 12, 2024

(30) Foreign Application Priority Data
Sep. 19, 2023 (JP) 2023-150813

(51) Int. Cl. A61F 2/89; D06M 23/14; D06M 23/16

(52) U.S. Cl. CPC A61F 2/89; D06M 23/14; D06M 23/16; A61F 2240/001; A61F 2250/0067

(57) ABSTRACT

A method for producing a living body indwelling object capable of stably fixing a porous structure to a stent. The method for producing a living body indwelling object includes disposing a porous structure fixing material and a porous structure having a knitted stitch structure on a stent extending in an axial direction and formed to be capable of inflating in a radial direction, and fixing the porous structure to the stent by melting the porous structure fixing material.

### CROSS-REFERENCES TO RELATED APPLICATIONS

This application claims priority to Japanese Application No. 2023-150813 filed on Sep. 19, 2023, the entire content of which is incorporated herein by reference.

### TECHNOLOGICAL FIELD

The present disclosure generally relates to a method for producing a living body indwelling object.

### BACKGROUND DISCUSSION

A stent is a medical device that is indwelt after being delivered to a lesion area in a body lumen such as a blood vessel by a stent delivery system, expands the lesion area such as a stenosis site or an occlusion site, and secures a lumen to treat various diseases caused by stenosis or occlusion of the body lumen.

Of the known medical devices and methods, each has certain advantages and disadvantages. There is an ongoing need to provide alternative medical devices as well as alternative methods for manufacturing and using medical devices.

### SUMMARY

A method for producing a living body indwelling object is disclosed.

(1) A method for producing a living body indwelling object, including: disposing a porous structure having a large number of penetrating voids and a porous structure fixing material on a stent extending in an axial direction and formed to be capable of expanding in a radial direction; and fixing the porous structure to the stent by melting the porous structure fixing material.

(2) In the method for producing a living body indwelling object according to (1), in the disposing, the porous structure is disposed on a surface of the porous structure fixing material after the porous structure fixing material is disposed on the stent.

(3) In the method for producing a living body indwelling object according to (1) or (2), in the disposing, the porous structure fixing material is disposed so as to form a gently bumped shape in a cross-sectional view.

(4) In the method for producing a living body indwelling object according to any one of (1) to (3), in the fixing, the porous structure is embedded in the porous structure fixing material.

(5) In the method for producing a living body indwelling object according to any one of (1) to (4), the porous structure fixing material is formed of a material selected from the group consisting of a polymer and a wax.

(6) In the method for producing a living body indwelling object according to any one of (1) to (5), a material constituting the porous structure fixing material has a melting point lower than a melting point of a material constituting the porous structure.

(7) In the method for producing a living body indwelling object according to any one of (1) to (6), in the disposing, the porous structure fixing material is disposed on a part of the stent.

(8) In the method for producing a living body indwelling object according to any one of (1) to (7), the stent includes a linear ring that forms a cylindrical outer periphery having gaps, and a plurality of link portions that connect the rings through the gaps, and in the disposing step, the porous structure fixing material is disposed on at least one of the plurality of link portions.

(9) In the method for producing a living body indwelling object according to any one of (1) to (8), a drug coating layer coated with a drug and an exposed portion not coated with the drug are formed on an outer surface of the stent, and in the disposing, the porous structure fixing material is disposed on the exposed portion.

(10) In the method for producing a living body indwelling object according to any one of (1) to (9), the porous structure is formed by jersey knitting.

(11) A method for producing a living body indwelling object for fixing a porous structure having a large number of penetrating voids to a stent extending in an axial direction and formed to be capable of expanding in a radial direction, the method including: pressing a porous structure fixing material and the porous structure disposed on the stent toward the stent by a pressing body; and melting the porous structure fixing material to weld the porous structure to the stent.

(12) In the method for producing a living body indwelling object according to (11), in a melting the porous structure fixing material, the porous structure fixing material is melted by a method selected from the group consisting of a laser, a light beam, heat, and ultrasounds.

(13) In the method for producing a living body indwelling object according to (11) or (12), the pressing body has a laser light transmission property, and in the melting the porous structure fixing material, the porous structure fixing material is irradiated with laser light via the pressing body.

(14) In the method for producing a living body indwelling object according to (11) or (12), in the melting the porous structure fixing material, the stent on which the porous structure fixing material and the porous structure are disposed by pressing using the pressing body that is heated.

(15) A stent extending in an axial direction and configured to be inflatable in a radial direction, the stent comprising: a plurality of wavy rings that forms a cylindrical outer periphery having gaps, and a plurality of link portions that connect the plurality of wavy rings through the gaps; and a porous structure fixed on at least one of the plurality of link portions.

### BRIEF DESCRIPTION OF THE DRAWINGS

FIG. 1 is a schematic plan view showing a stent delivery system including a living body indwelling object according to an embodiment of the disclosure.

FIG. 2 is a schematic plan view showing a reduced diameter state of the living body indwelling object according to the present embodiment.

FIG. 3 is a schematic plan view showing an increased diameter state of the living body indwelling object according to the present embodiment.

FIG. 4 is a partially enlarged view showing the living body indwelling object according to the present embodiment.

FIG. 5 is a partially enlarged view showing a portion A in FIG. 4.

FIG. 6 is a partially enlarged view showing a portion A in FIG. 5.

FIG. 7A is a cross-sectional view taken along a line 7A-7A in FIG. 6.

FIG. 7B is a cross-sectional view taken along a line 7B-7B in FIG. 6.

FIG. 8 is a schematic plan view showing a vicinity of a distal end of the living body indwelling object according to the present embodiment.

FIG. 9 is a partially enlarged view showing a portion A in FIG. 8.

FIG. 10 shows, by a two-dot chain line, a shape of a stent when a diameter is reduced, and shows, by a solid line, a shape of the stent when the diameter is increased.

FIG. 11 is a diagram showing a flowchart of a method for producing a living body indwelling object according to the present embodiment.

FIG. 12 is a diagram corresponding to FIG. 3 and shows a state in which a porous structure fixing material is disposed on a stent in the method for producing a living body indwelling object according to the present embodiment.

FIG. 13 is a cross-sectional view corresponding to FIG. 7A and shows a state in which a porous structure is disposed in the disposing step in the method for producing a living body indwelling object according to the present embodiment.

FIG. 14 is a cross-sectional view corresponding to FIG. 7A and shows a pressing step in the method for producing a living body indwelling object according to the present embodiment.

FIG. 15 is a cross-sectional view corresponding to FIG. 7A and shows a fixing step in the method for producing a living body indwelling object according to the present embodiment.

FIG. 16 is a diagram showing a state in which a pressing body and a core bar are removed from FIG. 15.

FIG. 17 is a diagram corresponding to FIG. 4 and shows a living body indwelling object according to a modification.

FIG. 18 is a schematic diagram showing a stent according to the modification.

FIG. 19 is a schematic diagram showing the stent according to the modification.

### DETAILED DESCRIPTION

Set forth below with reference to the accompanying drawings is a detailed description of embodiments of a method for producing a living body indwelling object. The following description does not limit the technical scope and meaning of terms described in the claims. A dimensional ratio in the drawings may be exaggerated for convenience of illustration and may be different from an actual ratio.

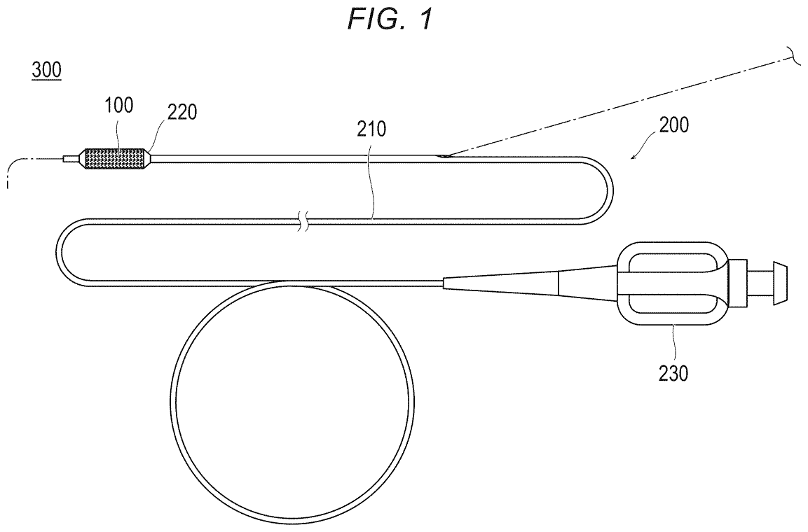

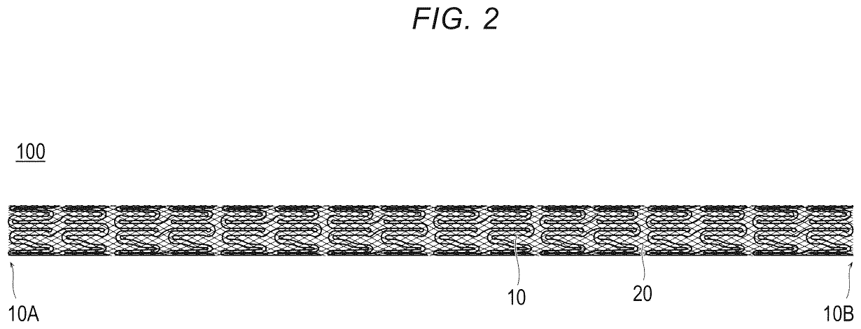

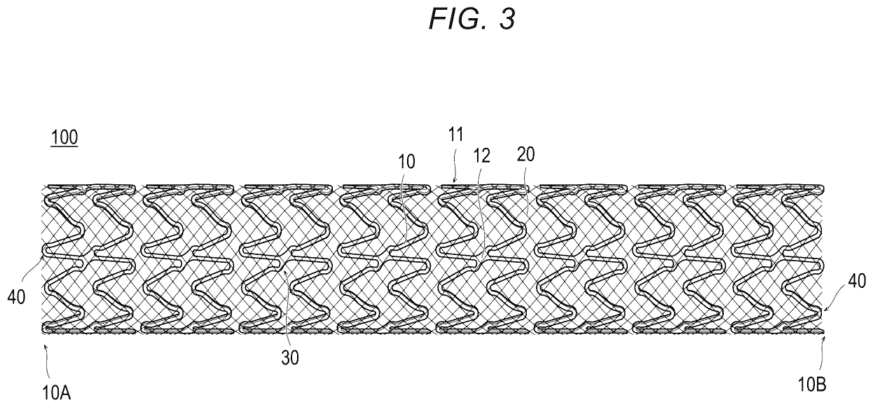

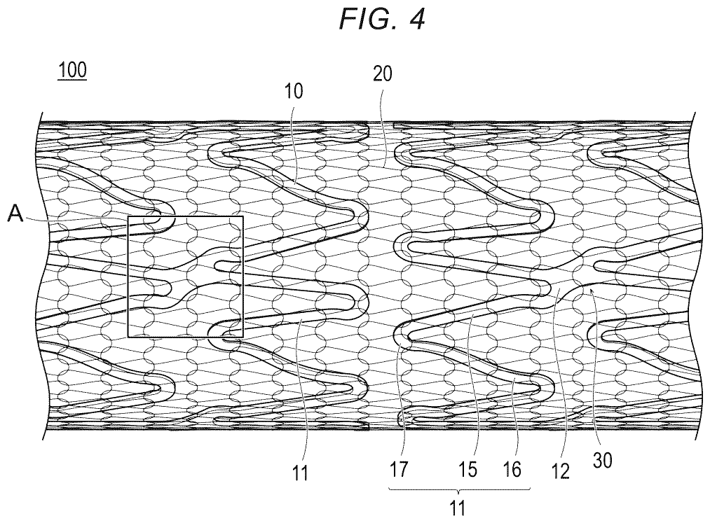

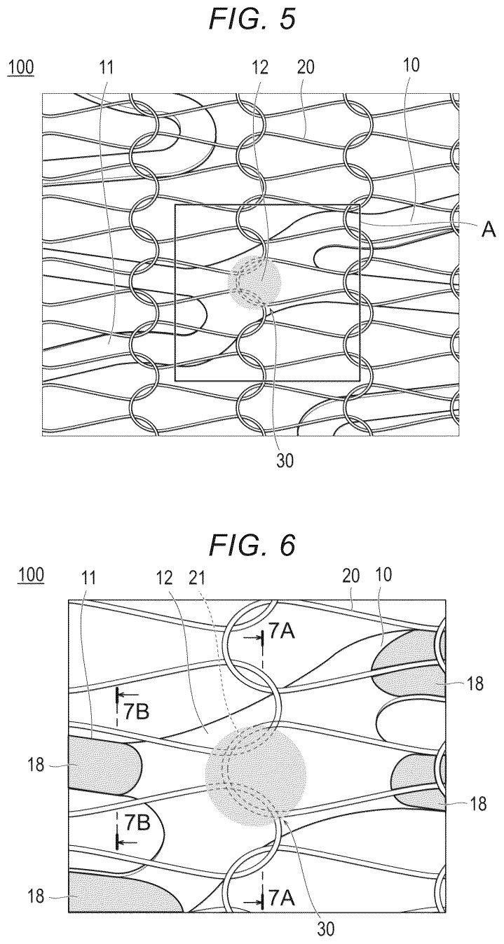

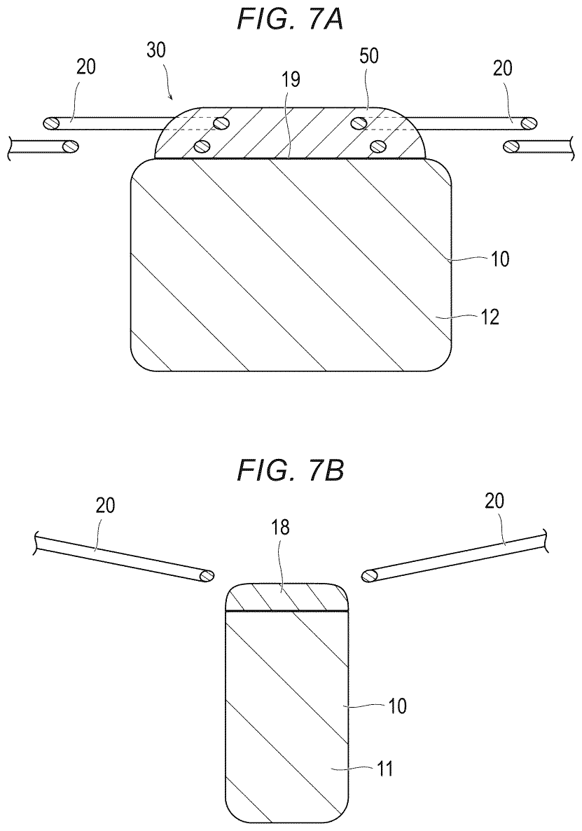

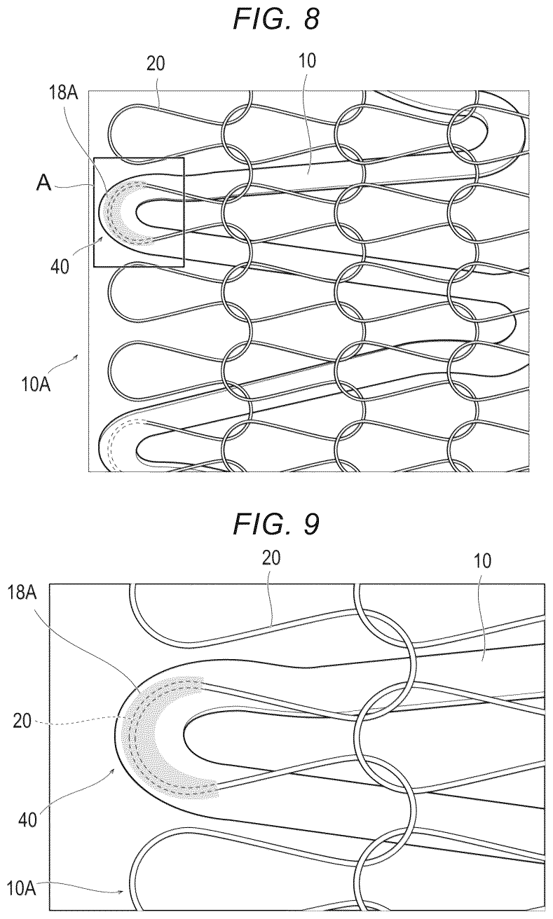

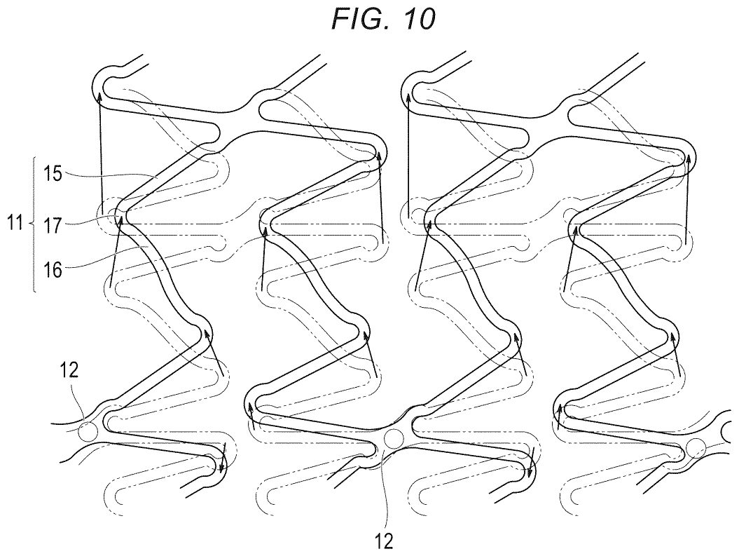

Hereinafter, a configuration of a living body indwelling object 100 according to the present embodiment will be described with reference to FIGS. 1 to 11. FIG. 1 is a schematic plan view showing a stent delivery system 300 including the living body indwelling object 100 according to the embodiment of the disclosure. FIG. 2 is a schematic plan view showing a reduced diameter state of the living body indwelling object 100 according to the present embodiment. FIG. 3 is a schematic plan view showing an increased diameter state of the living body indwelling object 100 according to the present embodiment. FIG. 4 is a partially enlarged view showing the living body indwelling object 100 according to the present embodiment. FIG. 5 is a partially enlarged view showing a portion A in FIG. 4. FIG. 6 is a partially enlarged view showing a portion A in FIG. 5. FIG. 7A is a cross-sectional view taken along a line 7A-7A in FIG. 6. FIG. 7B is a cross-sectional view taken along a line 7B-7B in FIG. 6. FIG. 8 is a schematic plan view showing a vicinity of a distal end of the living body indwelling object 100 according to the present embodiment. FIG. 9 is a partially enlarged view showing a portion A in FIG. 8. In FIG. 10, a two-dot chain line indicates a shape of a stent 10 when a diameter is reduced, and a solid line indicates the shape of the stent 10 when the diameter is increased.

In the present specification, a left-right direction in FIG. 2 is referred to as an “axial direction” of the living body indwelling object 100, and an upper-lower direction in FIG. 2 is referred to as a “radial direction” of the living body indwelling object 100. A side to be inserted into a living body is referred to as a “distal side”, and a side which is opposite to the distal side and on which an operator operates a medical device is referred to as a “proximal side”.

### Living Body Indwelling Object 100

As shown in FIG. 1, the living body indwelling object 100 according to the present embodiment constitutes the stent delivery system 300 by being disposed on an outer periphery of a balloon 220 of a balloon catheter 200.

The balloon catheter 200 is used to deliver the living body indwelling object 100 in an unexpanded state to a lesion area, and to expand and indwell the living body indwelling object 100 in the lesion area.

The balloon catheter 200 includes an elongated catheter main body portion 210, the balloon 220 provided at a distal end of the catheter main body portion 210, and a hub 230 fixed to a proximal end of the catheter main body portion 210. Since the balloon catheter 200 has a known configuration, a detailed description of the balloon catheter 200 will be omitted.

Hereinafter, the configuration of the living body indwelling object 100 will be described with reference to FIGS. 2 to 9. The living body indwelling object 100 according to the present embodiment is used to treat a stenosis site or an occlusion site occurring in a blood vessel, a bile duct, a trachea, an esophagus, a urethra, or other body lumens. The living body indwelling object 100 is a so-called balloon expansion type living body indwelling object which is disposed in a state of being crimped on the folded balloon 220 and is expanded and indwelt in the lesion area after being delivered to the lesion area.

As shown in FIGS. 2 to 9, the living body indwelling object 100 includes the stent 10 extending in the axial direction, a porous structure 20 disposed so as to cover an outer periphery of the stent 10, a fixing portion 30 at which the porous structure 20 is fixed to the stent 10, and both end fixing portions 40 at which the porous structure 20 is fixed to the stent 10 at a distal end 10A and a proximal end 10B of the stent 10.

The stent 10 extends in the axial direction (left-right direction in FIGS. 2 and 3) and includes the distal end 10A and the proximal end 10B. As shown in FIGS. 2 and 3, the stent 10 is formed so as to be capable of expanding (a state shown in FIG. 3) and contracting (a state shown in FIG. 2) in the radial direction. As shown in FIGS. 3 to 6, the stent 10 includes a plurality of linear rings (wavy rings) 11 that forms a cylindrical outer periphery having gaps, and link portions 12 that connect the plurality of rings 11 to each other through the gaps.

As shown in FIG. 4, there are a plurality of the wavy rings 11, and each wavy ring 11 includes first strut portions 15 formed of a straight line or a curved line, second strut portions 16 formed of a straight line or a curved line, and curved portions 17 formed between the first strut portions 15 and the second strut portions 16.

The rings 11 are sequentially disposed along the axial direction, and the rings 11 adjacent to each other in the axial direction are integrated by the link portion 12. Therefore, by increasing or decreasing the number of rings 11, the stent 10 having a desired length can be easily obtained.

As shown in FIGS. 6, 7A, and 7B, a drug coating layer 18 coated with a drug and an exposed portion 19 not coated with a drug are formed on an outer surface of the stent 10. The drug coated on the outer surface of the stent 10 is supported by a polymer to form the drug coating layer 18. The polymer is preferably a biodegradable polymer. In this case, after the stent 10 is indwelt in the living body, the drug is slowly released and the polymer is biodegraded, and thus stenosis at a stent indwelling site is reliably prevented from occurring again, and a polymer-derived inflammation is prevented.

Examples of the biodegradable polymer include at least one polymer selected from the group consisting of a polyester, an aliphatic polyester, a polyanhydride, a polyorthoester, a polycarbonate, a polyphosphazene, a polyphosphate ester, polyvinyl alcohol, a polypeptide, a polysaccharide, a protein, and cellulose, a copolymer obtained by freely copolymerizing monomers constituting the polymer, and a mixture of the polymers and/or the copolymers. Examples of the aliphatic polyester include polylactic acid (PLA), polyglycolic acid (PGA), and a lactic acid-glycolic acid copolymer (PLGA).

The drug coating layer 18 is disposed on the first strut portion 15 and the second strut portion 16 of the ring 11 shown in FIG. 4, and is disposed on an outer surface of the ring 11 as shown in FIG. 7B.

That is, the curved portion 17 of the ring 11 and the link portion 12 (portions where stress concentration and/or strain occurs due to expanding deformation) are not coated with the drug. The drug coating layer 18 is not formed on the curved portion 17 of the ring 11 and the link portion 12, and thus even when the stent 10 is inflated, the stress concentration and/or the strain is prevented from occurring in the drug coating layer 18.

A primer coating layer may be disposed between the drug coating layer 18 and the outer surface of the stent 10. A primer constituting the primer coating layer is selected in consideration of adhesiveness to the polymer contained in the drug coating layer 18 and adhesiveness to the outer surface of the stent 10, and presence of the primer coating layer improves separation resistance of the drug coating layer 18.

The stent 10 is made of a metal material or a polymer material. Here, the metal material used when the stent 10 is made of a metal material is not particularly limited, and a metal material commonly used for the stent 10 can be used. Specific examples of the metal material can include, for example, stainless steels such as SUS304, SUS316, SUS316L, SUS420J2, and SUS630, and tantalum, titanium, a nickel-titanium alloy, a tantalum-titanium alloy, a nickel-aluminum alloy, Inconel, gold, platinum, iridium, tungsten, and cobalt-based alloys such as a cobalt-chromium (Co—Cr) alloy.

In addition, the polymer material used when the stent 10 is made of a polymer material is not particularly limited, and a polymer material commonly used for the stent 10 can be used. Specific examples of the polymer material can include, for example, polyolefins such as polyethylene and polypropylene, aromatic polyesters such as polyethylene terephthalate, cellulose-based polymers such as cellulose acetate and cellulose nitrate, and fluorine-containing polymers such as polytetrafluoroethylene and a tetrafluoroethylene-ethylene copolymer.

The porous structure 20 is disposed so as to cover the outer periphery of the stent 10. The porous structure 20 can expand following the expansion of the stent 10. By disposing the porous structure 20 on the outer periphery of the stent 10, the porous structure 20 is urged against a blood vessel by the stent 10 when the stent 10 is expanded in the blood vessel. Then detachment of the porous structure 20 due to a pressure of a blood flow flowing in the blood vessel can be prevented. The porous structure 20 extends along the axial direction and may have a knitted stitch structure. Gaps of the knitted stitch structure of the porous structure 20 are preferably smaller than the gaps between the adjacent rings 11. According to this configuration, when the stent 10 is indwelt, it is possible to prevent peripheral embolus during the expansion of the stent 10 because stenosis objects overflowing from the gaps between the adjacent rings may be trapped by the knitted stitch structure of the porous structure 20. The porous structure 20 has a large number of penetrating voids. A size of the void is preferably smaller than an area of the gap between the rings 11 of the stent 10. As a result, it is possible to prevent scattering of a plaque and a thrombus due to the expansion of the stent 10. The size of the void is preferably larger than an area of a single blood cell contained in the blood. Accordingly, the blood cell can pass through the voids. Further the large number of penetrating voids may give the porous structure 20 a property of stretching. When the stent 10 is expanded in the radial direction, the porous structure 20 also may extend in a circumferential direction and easily follows the expansion of the stent 10.

The porous structure 20 includes a mesh and a molded product. The mesh includes a knitted product (knit) and a textile product (blade). The knitted product includes a knitted stich structure. When the porous structure 20 is the knitted product, the porous structure 20 is formed by jersey knitting. Since the jersey knitting is a known knitting method, a detailed description of the jersey knitting will be omitted. By forming the porous structure 20 by the jersey knitting, it is possible to prevent shortening of the porous structure 20 in the axial direction due to the expansion of the stent 10. When the porous structure 20 is a textile product, the textile product is formed by a known weaving method. In addition, the porous structure 20 may be formed by a molded product in which holes are formed in a cylindrical object formed by injection molding or the like.

A material constituting the porous structure 20 is not particularly limited, and examples of the material constituting the porous structure 20 can include: (1) a polymer selected from the group consisting of an aliphatic polyester, a polyester, a polyanhydride, a polyorthoester, a polycarbonate, a polyphosphazene, a polyphosphate ester, polyvinyl alcohol, a polypeptide, a polysaccharide, a protein, and cellulose; and (2) a copolymer composed of two or more monomers constituting the above (1). Here, the aliphatic polyester is not particularly limited, and examples of the aliphatic polyester can include polylactic acids (PLA) such as poly-L-lactic acid (PLLA), poly-D-lactic acid (PDLA), and poly-DL-lactic acid (PDLLA), polyglycolic acid (PGA), polyhydroxybutyric acid, polyhydroxyvaleric acid, polyhydroxypentanoic acid, polyhydroxyhexanoic acid, polyhydroxyheptanoic acid, poly(ε-caprolactone) (PCL), polytrimethylene carbonate, poly2,2-dimethyltrimethylene carbonate, polydioxanone, polybutyrolactone, polyvalerolactone, polymalic acid, polyethylene adipate, polyethylene succinate, polybutylene adipate, and polybutylene succinate. The polycarbonate is not particularly limited, and examples of the polycarbonate can include tyrosine-polycarbonate. Polymers other than the biodegradable polymer described above can also be used.

Further, the porous structure 20 may be made of a metal material. In this case, the same material as the metal material listed above for the stent 10 can be used.

When the porous structure 20 is formed by a knit or a blade, yarn diameter of the knit or the blade of the porous structure 20 is not particularly limited, and may be, for example, 20 μm.

In general, the fixing portion 30 is a position which is other than the distal end 10A and the proximal end 10B of the stent 10 and at which at least a part of the porous structure 20 is fixed to the stent 10. In the present specification, the distal end 10A of the stent 10 is defined as a “region which is a most distal end of the stent 10 and in which the link portion 12 disposed at the most distal end is not provided”. In addition, in the present specification, the proximal end 10B of the stent 10 is defined as a “region which is a most proximal end of the stent 10 and in which the link portion 12 disposed at the most proximal end is not provided”.

The fixing portion 30 is a portion where the porous structure 20 is fixed to the stent 10. In the present embodiment, the fixing portion 30 is made of a porous structure fixing material 50 that can be welded. By welding the porous structure fixing material 50 in the fixing portion 30, the porous structure 20 is embedded in the porous structure fixing material 50, and the porous structure 20 is fixed to the stent 10.

As a material used for the porous structure fixing material 50, the polymer materials listed above as the material constituting the porous structure 20 can be used. A wax may be used as the porous structure fixing material 50. As the porous structure fixing material 50, a biodegradable polymer is preferably used. According to this configuration, since the porous structure fixing material 50 is biodegradable, the polymer-derived inflammation is prevented.

In the present embodiment, as shown in FIGS. 3 to 6, the fixing portion 30 is provided at all locations of the link portions 12.

In the living body indwelling object 100 according to the present embodiment, as shown in FIGS. 6 and 7A, the fixing portion 30 is provided on the link portion 12. Here, as shown in FIG. 10, the link portion 12 is a location where a geometric change in the axial direction due to the expansion of the stent 10 is small. Therefore, tension of the porous structure 20 due to the expansion of the stent 10 is not big, and separation or breakage of the porous structure 20 can be suitably prevented.

As described above, the drug coating layer 18 is not provided on the link portion 12. Therefore, as shown in FIG. 7A, the fixing portion 30 is formed such that the porous structure 20 is directly fixed to the stent 10 on the exposed portion 19 of the stent 10 not coated with the drug. The porous structure fixing material 50 constituting the fixing portion 30 is directly fixed to the link portion 12 of the stent 10 as shown in FIG. 7A. According to this configuration, as compared with a configuration in which the porous structure fixing material 50 constituting the fixing portion 30 is fixed to the stent 10 via the drug coating layer 18, it is possible to improve adhesive strength of the porous structure 20 to the stent 10 when the porous structure fixing material 50 is welded.

In the case of a configuration in which the porous structure fixing material 50 is fixed to the stent 10 via the drug coating layer 18, for example, there is an effect that an inflammation reaction derived from the porous structure fixing material 50 is prevented by disposing the drug coating layer 18 in the vicinity of the porous structure fixing material 50.

The fixing portion 30 is formed such that the porous structure 20 is directly fixed to the stent 10 in the exposed portion 19 of the stent 10 not coated with the drug. As a result, even when the fixing portion 30 is welded by heating, since the drug coating layer 18 is not present, an effect of the drug is not lost. A configuration in which the porous structure fixing material 50 is fixed to the stent 10 via the drug coating layer 18 can be adopted when the fixing portion 30 is formed by a method without heating, for example, an adhesive or the like or with little influence of heating.

As shown in FIG. 6, in the fixing portion 30, a mesh loop portion 21 knitted along the circumferential direction of the porous structure 20 (upper-lower direction in FIG. 6) is fixed to the stent 10. At least the loop portion 21 is included in a fixed location, and intersections with the adjacent meshes may be included. According to this configuration, since the loop portion 21 extending along the circumferential direction is fixed to the stent 10, a long distance of the porous structure 20 can be embedded in the porous structure fixing material 50, and a fixing force can be improved.

As shown in FIGS. 8 and 9, at the both end fixing portions 40, the porous structure 20 is fixed to the stent 10 at the distal end 10A and the proximal end 10B of the stent 10. In particular, as shown in FIGS. 8 and 9, it is preferable that the both end fixing portions 40 are provided at a curved portion 18A at the distal end 10A of the stent 10 and a curved portion at the proximal end 10B of the stent 10.

A method for fixing the porous structure 20 to the stent 10 at the both end fixing portions 40 is the same as the method for fixing the porous structure 20 to the stent 10 at the fixing portion 30 described above, and thus a description of the method for fixing the porous structure 20 at the both ends fixing portions 40 will be omitted.

Here, in the living body indwelling object 100 according to the present embodiment, since the both end fixing portions 40 are present, it is possible to prevent the porous structure 20 from being detached from the stent 10 even in a case where curving in the lesion area is strong when the living body indwelling object 100 is delivered to the lesion area.

### Method for Producing Living Body Indwelling Object 100

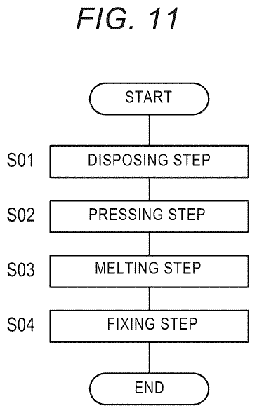

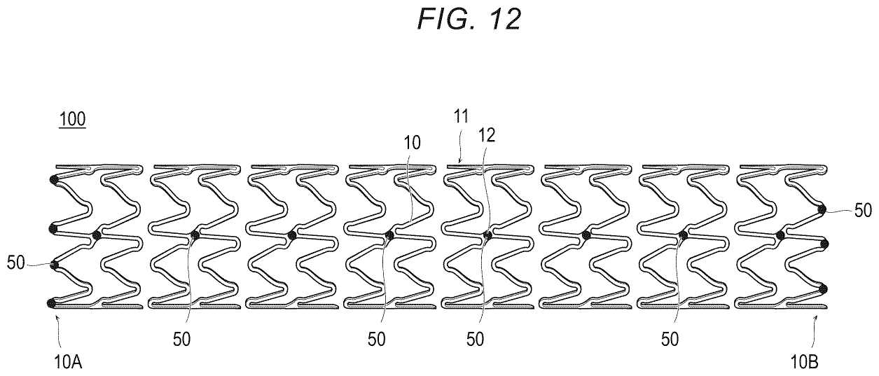

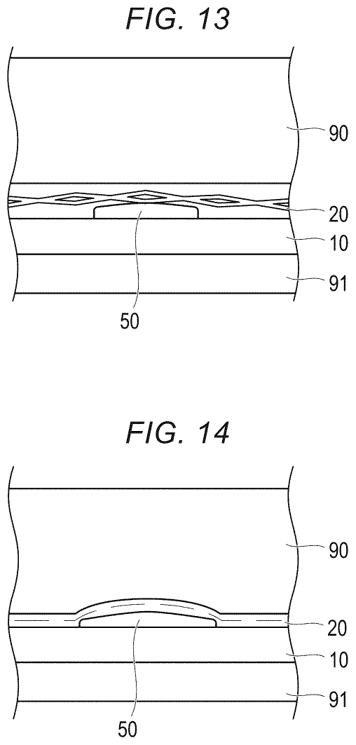

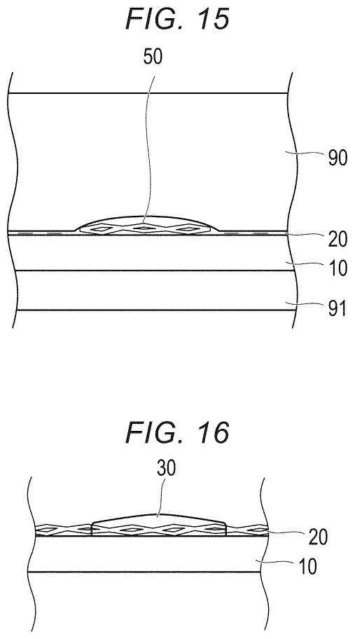

Next, a method for producing the living body indwelling object 100 will be described with reference to FIGS. 11 to 15. FIG. 11 is a diagram showing a flowchart of the method for producing the living body indwelling object 100 according to the present embodiment. FIG. 12 is a diagram corresponding to FIG. 3, and shows a state in which the porous structure fixing material 50 is disposed on the stent 10 in a disposing step S01 in the method for producing the living body indwelling object 100 according to the present embodiment. FIG. 13 is a cross-sectional view corresponding to FIG. 7A, and shows a state in which the porous structure 20 is disposed in the disposing step S01 in the method for producing the living body indwelling object 100 according to the present embodiment. FIG. 14 is a cross-sectional view corresponding to FIG. 7A, and shows a pressing step S02 in the method for producing the living body indwelling object 100 according to the present embodiment. FIG. 15 is a cross-sectional view corresponding to FIG. 7A, and shows a fixing step S04 in the method for producing the living body indwelling object 100 according to the present embodiment. In FIGS. 13 to 15, an upper half in the circumferential direction is shown.

In general, as shown in FIG. 11, the method for producing the living body indwelling object 100 according to the present embodiment includes the disposing step S01, the pressing step S02, a melting step S03, and the fixing step S04. Hereinafter, each step will be described in detail.

### Disposing Step S01

First, the disposing step S01 will be described. In the disposing step S01, as shown in FIG. 12, first, the porous structure fixing material 50 is disposed on a part of the stent 10. The porous structure fixing material 50 is disposed on an outer periphery of the stent 10. A method for disposing the porous structure fixing material 50 on the stent 10 is not particularly limited, and the porous structure fixing material 50 can be disposed by, for example, coating.

More specifically, the porous structure fixing material 50 is disposed on the link portion 12, the distal end 10A, and the proximal end 10B of the stent 10. Here, as shown in FIG. 12, the link portion 12 is a location where a geometric change in the axial direction due to the expansion of the stent 10 is small. Therefore, tension of the porous structure 20 due to the expansion of the stent 10 is not big, and separation or breakage of the porous structure 20 can be suitably prevented. Since the porous structure fixing material 50 is disposed on the link portion 12, the distal end 10A, and the proximal end 10B of the stent 10, when the porous structure 20 is fixed to the stent 10 in the fixing step S04 to be described later, it is possible to prevent the porous structure 20 from being deformed in a manner of spreading outward in the radial direction and becoming bulky. Therefore, it is possible to prevent the porous structure 20 from being deformed in the manner of spreading outward in the radial direction, and to prevent deliverability of the living body indwelling object 100 from being lowered. When the drug coating layer 18 is not provided on the link portion 12 as described above, the porous structure fixing material 50 is disposed on the exposed portion 19 of the stent 10. Therefore, as described above, as compared with a configuration in which the porous structure fixing material 50 is fixed to the stent 10 via the drug coating layer 18, it is possible to improve adhesive strength of the porous structure 20 to the stent 10 when the porous structure fixing material 50 is welded.

After the porous structure fixing material 50 is disposed on the stent 10, the porous structure fixing material 50 has a gently bumped shape in a cross-sectional view as shown in FIG. 13. The words “gently bumped shape” could refer to a state in which a slope of a central portion is gentler than a slope of a peripheral portion in a cross-sectional view. Therefore, when the stent 10 on which the porous structure fixing material 50 is disposed is deformed, it is possible to prevent the separation of the porous structure fixing material 50 due to strain or stress concentration in the porous structure fixing material 50. Further, when the living body indwelling object 100 is delivered to the lesion area, it is possible to prevent the porous structure fixing material 50 from being caught by an intravascular wall, a bifurcated portion, an end portion of the catheter, or the like.

After the porous structure fixing material 50 is disposed on the stent 10, the porous structure 20 is disposed on an outer surface of the porous structure fixing material 50 as shown in FIG. 13.

In the method for producing the living body indwelling object 100 according to the present embodiment, the porous structure 20 is disposed on the outer surface of the porous structure fixing material 50 after the porous structure fixing material 50 is disposed on the stent 10, and thus when the porous structure fixing material 50 is melted in the melting step S03, the porous structure 20 can be suitably fixed to the stent 10. The method in which the porous structure 20 is first disposed over the surface of the stent 10 and the porous structure fixing material 50 is disposed on the outer surface of the porous structure 20 can be implemented by adjusting a viscosity when the porous structure fixing material 50 can be, for example, a polymer solution or an adhesive, so that the polymer solution or the adhesive flows around to a lower side of the porous structure, and the polymer solution or the adhesive does not wet and spread in a lateral direction due to a capillary phenomenon.

### Pressing Step S02

Next, the pressing step S02 will be described. In the pressing step S02, first, as shown in FIG. 13, a pressing body 90 is prepared on an outer periphery of the porous structure 20, and a core bar 91 is prepared on an inner periphery of the stent 10. The core bar 91 functions as a receiving tool when the pressing body 90 presses the porous structure 20, the porous structure fixing material 50, and the stent 10. The pressing body 90 has a laser light transmission property. A material for the pressing body 90 can be, for example, silicone rubber or fluororubber.

Next, in the pressing step S02, as shown in FIG. 14, the pressing body 90 presses the porous structure 20, the porous structure fixing material 50, and the stent 10 from the outer periphery to the inner periphery toward the core bar 91. As a result, tension toward the stent 10 is generated on the porous structure 20.

### Melting Step S03

Next, the melting step S03 will be described. In the melting step S03, the porous structure fixing material 50 is irradiated with laser light to melt the porous structure fixing material 50. The laser light is emitted from radially outward of the pressing body 90 toward the porous structure fixing material 50 via the pressing body 90. A wavelength of the emitted laser light can be, for example, 800 nm to 10000 nm. Here, since the pressing body 90 has the laser light transmission property, the emitted laser passes through the pressing body 90 and is emitted to the porous structure fixing material 50. According to this method, since the porous structure fixing material 50 can be melted in a relatively short time (for example, within several seconds), a time of the melting step S03 can be shortened.

The material constituting the porous structure fixing material 50 preferably has a melting point lower than that of the material constituting the porous structure 20. According to this configuration, in the fixing step S04 to be described later, the porous structure 20 is suitably embedded in the porous structure fixing material 50.

### Fixing Step S04

Next, the fixing step S04 will be described. The fixing step S04 is a step performed continuously with the melting step S03. That is, when the porous structure fixing material 50 at one location is melted in the melting step S03, the fixing step S04 is continuously performed at the same location.

In the fixing step S04, as shown in FIG. 15, the porous structure 20 in which the tension is generated toward the inner periphery in the pressing step S02 is embedded in the porous structure fixing material 50 melted in the melting step S03. By embedding the porous structure 20 in the porous structure fixing material 50 in such a manner, welding strength of the porous structure 20 to the stent 10 is improved. Through the above steps, the porous structure 20 is fixed to the stent 10 by the porous structure fixing material 50.

FIG. 16 is a diagram showing a state in which the pressing body 90 and the core bar 91 are removed. The porous structure fixing material 50 is formed as the fixing portion 30 at which the porous structure 20 is fixed to the stent 10 by the above steps.

As described above, the method for producing the living body indwelling object 100 according to the present embodiment may include the disposing step S01 of disposing the porous structure 20 having a large number of penetrating voids and the porous structure fixing material 50 on the stent 10 extending in the axial direction and formed to be capable of expanding in the radial direction. The method may include the fixing step S04 of fixing the porous structure 20 to the stent 10 by melting the porous structure fixing material 50. According to this production method, after the porous structure fixing material 50 and the porous structure 20 are preferably disposed on the stent 10 in the disposing step S01, the porous structure 20 is fixed to the stent 10 by melting the porous structure fixing material 50 in the fixing step S04, and thus the porous structure 20 can be stably fixed to the stent 10.

In the disposing step S01, after the porous structure fixing material 50 is disposed on the stent 10, the porous structure 20 is preferably disposed on the surface of the porous structure fixing material 50. According to this production method, when the porous structure fixing material 50 is melted in the melting step S03, the porous structure 20 can be suitably fixed to the stent 10.

In the disposing step S01, the porous structure fixing material 50 is preferably disposed so as to form the gently bumped shape in the cross-sectional view. According to this production method, when the stent 10 on which the porous structure fixing material 50 is disposed is deformed, it is possible to prevent the separation of the porous structure fixing material 50 due to strain or stress concentration in the porous structure fixing material 50.

In the fixing step S04, the porous structure 20 is preferably embedded in the porous structure fixing material 50. According to this production method, the welding strength of the porous structure 20 to the stent 10 is improved.

The porous structure fixing material 50 is preferably formed of a material selected from the group consisting of a polymer and a wax. According to this production method, a suitable melting method can be selected according to the material of the porous structure fixing material 50.

The material constituting the porous structure fixing material 50 preferably has a melting point lower than that of the material constituting the porous structure 20. According to this production method, in the fixing step S04, the porous structure 20 is suitably embedded in the porous structure fixing material 50.

In the disposing step S01, the porous structure fixing material 50 is preferably disposed on a part of the stent 10. According to this production method, it is possible to prevent the porous structure 20 from being deformed in the manner of spreading outward in the radial direction and becoming bulky. Therefore, it is possible to prevent the porous structure 20 from being deformed in the manner of spreading outward in the radial direction, and to prevent deliverability of the living body indwelling object 100 from being lowered.

In addition, the stent 10 preferably includes the linear rings 11 that form the cylindrical outer periphery having gaps, and the plurality of link portions 12 that connect the rings 11 through the gap, and in the disposing step S01, the porous structure fixing material 50 is preferably disposed on at least one of the plurality of link portions 12. According to this production method, it is possible to prevent the porous structure 20 from being deformed in the manner of spreading outward in the radial direction and becoming bulky. Therefore, it is possible to prevent the porous structure 20 from being deformed in the manner of spreading outward in the radial direction, and to prevent deliverability of the living body indwelling object 100 from being lowered.

The drug coating layer 18 coated with the drug and the exposed portion 19 not coated with the drug are preferably formed on the outer surface of the stent 10, and the porous structure fixing material 50 is disposed on the exposed portion 19 in the disposing step S01. According to this production method, adhesive strength of the porous structure 20 to the stent 10 when the porous structure fixing material 50 is welded can be improved.

The porous structure 20 is preferably formed by the jersey knitting. According to this production method, it is possible to prevent the shortening of the porous structure 20 in the axial direction due to the expansion of the stent 10.

In the method for producing the living body indwelling object 100 for fixing the porous structure 20 having the knitted stitch structure to the stent 10 extending in the axial direction and formed to be capable of inflating in the radial direction, the porous structure fixing material 50 disposed on the stent 10 and the porous structure 20 are preferably pressed toward the stent 10 by the pressing body 90. And then the porous structure fixing material 50 is melted to fix the porous structure 20 to the stent 10. According to this production method, the porous structure 20 can be stably fixed to the stent 10.

In a step of melting the porous structure fixing material 50, the porous structure fixing material 50 is preferably melted by a method selected from the group consisting of a laser, a light beam, heat, and ultrasounds. According to this production method, the porous structure fixing material 50 can be melted by a suitable method.

The pressing body 90 preferably has the laser light transmission property, and in the step of melting the porous structure fixing material 50, the porous structure fixing material 50 is preferably irradiated with laser light via the pressing body 90. According to this production method, since the porous structure fixing material 50 can be melted in a relatively short time (for example, within several seconds), the time of the melting step S03 can be shortened.

Although the method for producing the living body indwelling object 100 according to the disclosure is described above through the embodiment, the disclosure is not limited to the method described in the embodiment, and can be appropriately changed based on the description of the claims.

For example, in the above-described embodiment, the fixing portion 30 is provided at all locations of the link portions 12, but the fixing portion 30 may be provided at a part of all the link portions 12. According to this configuration, it is possible to reduce a use amount of the porous structure fixing material 50 constituting the fixing portion 30, and to prevent the porous structure 20 from being deformed in the manner of spreading outward in the radial direction and becoming bulky.

In the above-described embodiment, the fixing portion 30 is provided on the link portion 12. However, the fixing portion 30 may be provided on the first strut portion 15 or the second strut portion 16. According to this configuration, it is also possible to prevent the porous structure 20 from being deformed in the manner of spreading outward in the radial direction, and to prevent the deliverability of the living body indwelling object 100 from being lowered.

In the above-described embodiment, the fixing portion 30 is provided on the link portion 12. However, the fixing portion 30 may be provided on the link portion 12 and the strut. According to this configuration, since the fixing portion 30 is provided on the link portion 12 and the strut, the strength is further improved.

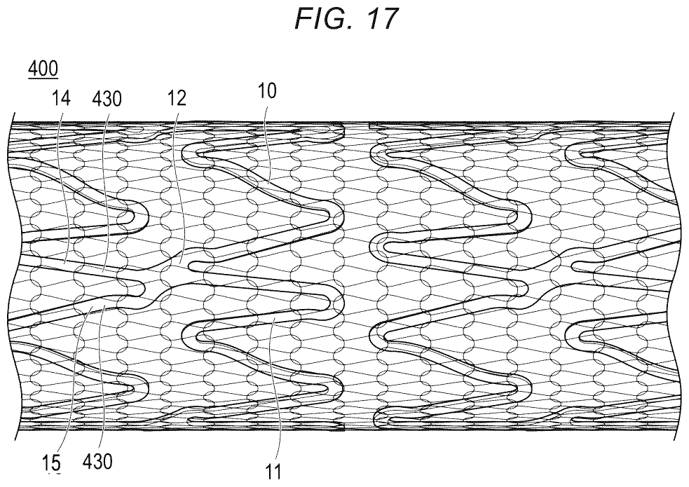

In the above-described embodiment, the fixing portion 30 is provided on the link portion 12. However, as shown in FIG. 17, a fixing portion 430 of a living body indwelling object 400 according to a modification may be provided in a third strut portion 14 and/or the fourth strut portion (first strut portion) 15 which are adjacent to one side (for example, a left side in FIG. 17) of the link portion 12 in the axial direction and which are provided as a pair in the circumferential direction (upper and lower in FIG. 17). FIG. 17 shows an example in which the third strut portion 14 and the fourth strut portion 15 are provided with the fixing portion 430. According to this configuration, since the third strut portion 14 and the fourth strut portion 15 are provided close to the link portion 12, a geometric change in the axial direction before and after the expansion is smaller than in other struts, and the separation or breakage of the porous structure 20 can be reduced. In addition, when the fixing portion 430 is provided on the third strut portion 14 or the second strut portion 16, the separation or breakage of the porous structure 20 can be further reduced as compared with the case where the fixing portion 430 is provided on both the third strut portion 14 and the second strut portion 16.

In the above-described embodiment, the drug coating layer 18 coated with the drug is formed on the outer surface of the stent 10, but the drug coating layer 18 may not be formed on the outer surface of the stent 10, and the fixing portion 30 may be provided on a part of the link portion 12 or the strut.

Further, in the above-described embodiment, the living body indwelling object 100 is a balloon expandable type living body indwelling object. However, the living body indwelling object 100 may be a so-called self-expandable stent which is compressed in a central axis direction at the time of inserting into a living body and inflates outward to restore a shape before the compression at the time of indwelling into the living body. When the stent 10 is the self-expandable stent, a superelastic alloy such as a nickel-titanium alloy is preferable since a restoring force to an original shape is required.

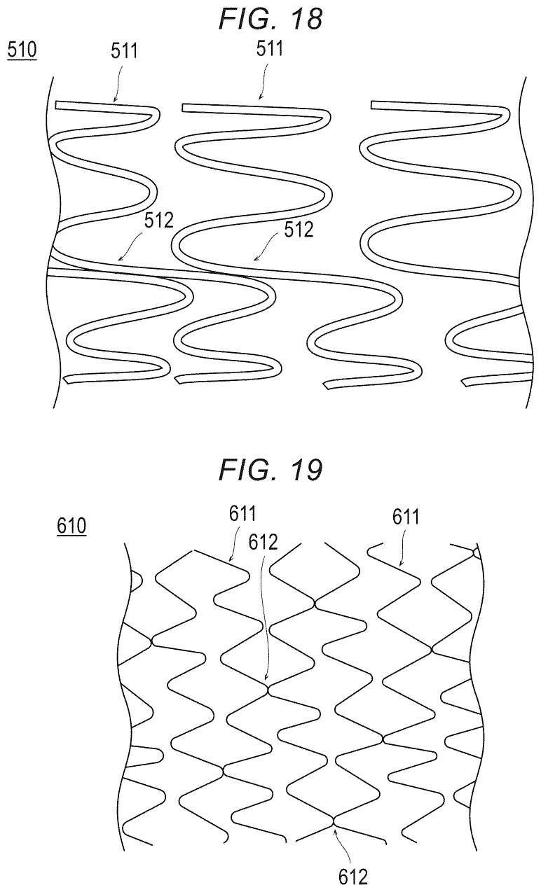

The shape of the stent is not particularly limited, and the stent may have a shape as shown in FIGS. 18 and 19. For example, a stent 510 shown in FIG. 18 includes rings 511 and link portions 512. In addition, a stent 610 shown in FIG. 19 includes rings 611 and link portions 612.

Further, in the above-described embodiment, the pressing body 90 presses the porous structure 20 to the stent 10 in the pressing step S02, so that the porous structure 20 is embedded in the porous structure fixing material 50 in the fixing step S04. However, when the pressing body 90 is not provided and the porous structure 20 deflates by itself, the porous structure 20 may be embedded in the porous structure fixing material 50 in the fixing step S04.

In the above-described embodiment, after the porous structure fixing material 50 is disposed on the stent 10, the porous structure fixing material 50 has the gently bumped shape in the cross-sectional view. However, after the porous structure fixing material 50 is disposed on the stent 10, the porous structure fixing material 50 may not have the gently bumped shape in the cross-sectional view.

In the above-described embodiment, in the fixing step S04, the porous structure 20 is embedded in the porous structure fixing material 50. However, in the fixing step S04, the porous structure 20 may not be completely embedded in the porous structure fixing material 50, and the porous structure 20 being not completely embedded in the porous structure fixing material 50 is included in the invention as long as the porous structure 20 is fixed to the stent 10.

In the above-described embodiment, the porous structure fixing material 50 is disposed on the link portion 12, the distal end 10A, and the proximal end 10B of the stent 10. However, the porous structure fixing material 50 may be disposed at any position of the stent 10.

In the above-described embodiment, in the melting step S03, the porous structure fixing material 50 is melted by being irradiated with a laser, but the porous structure fixing material 50 may be melted by being irradiated with a light beam or ultrasounds.

In the above-described embodiment, in the melting step S03, the porous structure fixing material 50 is melted by being irradiated with a laser. However, the stent 10 on which the porous structure fixing material 50 and the porous structure 20 are disposed by pressing using the pressing body 90, which may be heated. A heating temperature at this time is equal to or higher than the melting point of the porous structure fixing material 50. According to this production method, a processing time can be shortened.

The detailed description above describes to a method for producing a living body indwelling object. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for producing a living body indwelling object, comprising:
disposing a porous structure having a large number of penetrating voids and a porous structure fixing material on a stent, the stent extending in an axial direction and configured to be inflatable in a radial direction; and
fixing the porous structure to the stent by melting the porous structure fixing material.

2. The method for producing a living body indwelling object according to claim 1, wherein the porous structure includes a mesh having a knitted stitch structure, and the porous structure fixing material includes a mesh fixing material.

3. The method for producing a living body indwelling object according to claim 1, further comprising:
disposing the porous structure on a surface of the porous structure fixing material after the porous structure fixing material is disposed on the stent.

4. The method for producing a living body indwelling object according to claim 1, further comprising:
disposing the porous structure fixing material to form a gently bumped shape in a cross-sectional view.

5. The method for producing a living body indwelling object according to claim 1, further comprising:
embedding the porous structure in the porous structure fixing material.

6. The method for producing a living body indwelling object according to claim 1, wherein
the porous structure fixing material is formed of a material selected from a group consisting of a polymer and a wax.

7. The method for producing a living body indwelling object according to claim 1, wherein
a material constituting the porous structure fixing material has a melting point lower than a melting point of a material constituting the porous structure.

8. The method for producing a living body indwelling object according to claim 1, further comprising:
disposing the porous structure fixing material on a part of the stent.

9. The method for producing a living body indwelling object according to claim 1, wherein
the stent includes a plurality of linear rings that forms a cylindrical outer periphery having gaps, and a plurality of link portions that connect the plurality of rings through the gaps, and the method further comprises:
disposing the porous structure fixing material on at least one of the plurality of link portions.

10. The method for producing a living body indwelling object according to claim 1, wherein
a drug coating layer coated with a drug and an exposed portion not coated with the drug are formed on an outer surface of the stent, and the method further comprises:
disposing the porous structure fixing material on the exposed portion not coated with the drug formed on the outer surface of the stent.

11. The method for producing a living body indwelling object according to claim 1, further comprising:
forming the porous structure by jersey knitting.

12. A method for producing a living body indwelling object for fixing a porous structure having a large number of penetrating voids to a stent extending in an axial direction and formed to be capable of inflating in a radial direction, the method comprising:
pressing a porous structure fixing material and the porous structure disposed on the stent toward the stent by a pressing body; and
melting the porous structure fixing material to fix the porous structure to the stent.

13. The method for producing a living body indwelling object according to claim 12, wherein
the porous structure includes a mesh having a knitted stitch structure, and the porous structure fixing material includes a mesh fixing material.

14. The method for producing a living body indwelling object according to claim 12, wherein
melting the porous structure fixing material by a method selected from a group consisting of a laser, a light beam, heat, and ultrasounds.

15. The method for producing a living body indwelling object according to claim 12, wherein
the pressing body has a laser light transmission property, and the method further comprises:
irradiating the porous structure fixing material is irradiated with laser light via the pressing body.

16. The method for producing a living body indwelling object according to claim 12, further comprising:
pressing the stent on which the porous structure fixing material and the porous structure are disposed with the pressing body that is heated.

17. A stent extending in an axial direction and configured to be inflatable in a radial direction, the stent comprising:
a plurality of wavy rings that forms a cylindrical outer periphery having gaps, and a plurality of link portions that connect the plurality of wavy rings through the gaps; and
a porous structure fixed on at least one of the plurality of link portions.

18. The stent according to claim 17, wherein the porous structure is melted onto the stent with a porous structure fixing material, the porous structure fixing material selected from a group consisting of a polymer and a wax.

19. The stent according to claim 17, wherein each of the plurality of the wavy rings includes first strut portions formed of a straight line or a curved line, second strut portions formed of a straight line or a curved line, and curved portions formed between the first strut portions and the second strut portions.

20. The stent according to claim 17, wherein the porous structure includes a mesh having a knitted stitch structure, and the porous structure fixing material includes a mesh fixing material.
